(19) United States
(12) Patent Application Publication
(10) Pub. No.: US 2008/0025933 A1
(43) Pub. Date: Jan. 31, 2008

(54) Elastic cationic polyurethanes

(75) Inventor: Nathalie Mougin, Paris (FR)

Correspondence Address: NIXON & VANDERHYE, PC, 901 NORTH GLEBE ROAD, 11TH FLOOR, ARLINGTON, VA, 22203, US

(73) Assignee: L'OREAL, Paris (FR)

(21) Appl. No.: 11/905,236

(22) Filed: Sep. 28, 2007

Related U.S. Application Data
(62) Division of application No. 10/398,904, filed on Oct. 1, 2003.

(30) Foreign Application Priority Data
Oct. 17, 2000 (FR) 00/13264

(51) Int. Cl. A61K 8/87; A61Q 19/00; A61Q 3/02; A61Q 5/00

(52) U.S. Cl. 424/61; 424/70.17; 424/78.03; 528/59

(57) ABSTRACT

The invention concerns novel cationic polyurethanes with elastic character, that is having immediate recovery ranging between 5% and 95%, consisting essentially (a1) of cationic units derived from at least a tertiary amine having at least two functions reactive to labile hydrogen, said tertiary amine being at least partly neutralised, (a2) non-ionic units derived from non-ionic polymer s bearing at their ends functions reactive to labile hydrogen and having a glass transition temperature (Tg), measured by differential heat content analysis, less than 10° C., optionally (a3) non-ionic units derived from non-ionic monomer compounds containing at least two functions reactive to labile hydrogen, and (b) units derived from at least a diisocyanate, and cosmetic compositions containing said polymers.

This application is a divisional of application Ser. No. 10/398,904, filed Oct. 1, 2003 (pending), which is a U.S. National Phase of International Application No. PCT/FR01/03157, filed Oct. 12, 2001, which designated the U.S. and claims benefit of FR 00/13264, filed Oct. 17, 2000, the entire contents of each of which is hereby incorporated by reference in this application.

The present invention relates to new elastic cationic polyurethanes and their use in cosmetic compositions.

A large amount of research has always been done for cosmetics on the formation of deposits and films with elastic properties. Most parts of the human body on which cosmetic applications are likely to be made, such as the skin, lips, hair, eyelashes and finger and toe nails are subject to large deformations and mechanical stresses. Cosmetic films and applications must be capable of resist these stresses and must be able to follow these deformations without breaking.

The use of polyurethanes in cosmetics has been known for a long time and is described for example in patents WO94/13724 and EP 0 619 111.

However, the polyurethanes divulged in these documents have vitreous transition temperatures (Tg) greater than ambient temperature (20° C.), in other words they are in the vitreous state at ambient temperature and they form brittle films that are unacceptable for a cosmetic application.

Undoubtedly, there are physiologically acceptable polymers with low vitreous transition temperatures, for example such as acrylic polymers, but these polymers usually form very sticky deposits, and this is undesirable for most cosmetic applications.

The applicant discovered, surprisingly, a new group of physiologically acceptable polyurethanes that form films that do not stick, are not brittle and are capable of plastic and elastic deformations. These attractive viscoelastic properties are due to the presence of long macromolecular patterns in the polymer with a relatively low vitreous transition temperature and consequently, they are not in the vitreous state at ambient temperature.

Consequently, the purpose of the present invention is elastic cationic polyurethanes composed essentially of

(a1) cationic patterns derived from at least one tertiary amine with at least two labile hydrogen reactive functions, the said amine being at least partially neutralised,

(a2) non-ionic patterns derived from non-ionic polymers carrying reactive labile hydrogen functions at their ends with a vitreous transition temperature (Tg) measured by differential scanning calorimetry, less than 10° C., possibly

(a3) non-ionic patterns derived from non-ionic monomer compounds containing at least two labile hydrogen functions, and

(b) patterns derived from at least one diisocyanate.

Another purpose of the invention is the use of elastic cationic polyurethanes described above in cosmetic compositions in order to improve the viscoelastic properties of cosmetic applications and films obtained from these compositions.

In particular, another purpose is the use of these polyurethanes in lacquers and hair compositions, and in nail varnish and make-up compositions.

Another purpose of the invention is cosmetic compositions containing the elastic cationic polyurethanes described above.

Due to their cationic charge, elastic polyurethanes according to this invention have the advantage of an excellent affinity for keratinic substrates such as hair, finger and toe nails, and the corneal layer of the epidermis, to which keratin applies a negative charge.

The use of elastic cationic polyurethanes according to this invention in lacquers and hair compositions makes hair more supple, in other words it makes its behaviour more naturally elastic than is possible with usual fixing polymers.

These polyurethanes are used to cover finger and toe nails with a glossy protective film resistant to mechanical aggression. Their use in nail varnish improves the resistance of hair varnish to shocks and retards scaling.

The cationic polyurethanes described above can also be used to improve the behaviour of make-up compositions for the skin, lips and superficial body growth. Make-up products containing these polymers bond well to the skin and superficial body growth, and the coatings obtained follow deformations of the keratinic substrates and do not dry the skin.

Non-sticking products are obtained for all these applications.

As mentioned above, elastic cationic polyurethanes according to the present invention are composed essentially of three types of patterns

(a1) cationic patterns derived from at least one tertiary amine with at least two labile hydrogen reactive functions, the said amine being at least partially neutralised, and

(a2) non-ionic patterns derived from non-ionic polymers carrying reactive labile hydrogen functions at their ends with a vitreous transition temperature (Tg) measured by differential scanning calorimetry, less than 10° C., possibly

(b) patterns derived from at least one diisocyanate.

The expression “Reactive labile hydrogen functions” means functions capable of forming covalent links with isocyanate functions of compounds forming patterns (b), after the departure of a hydrogen atom. For example, these functions include hydroxyl groups, primary amine groups (—NH₂), secondary amine groups (—NHR), or thiol groups (—SH).

Polycondensation of compounds carrying these reactive labile hydrogen functions with diisocyanates gives polyurethanes, polyureas and polythio-urethanes, depending on the nature of reactive functions carrying the labile hydrogen (—OH, —NH₂, —NHR or —SH) respectively. In this present application, all these polymers are grouped under the term “polyurethanes”, for simplification purposes.

When tertiary amines forming patterns (a1) contain more than two labile hydrogen functions, the polyurethanes obtained have a ramified structure.

In one preferred embodiment of polyurethanes according to the present invention, tertiary amines forming the cationic patterns (a1) only have two reactive labile hydrogen functions and consequently the polyurethanes obtained by polycondensation have an essentially linear structure.

Obviously, a mix of dysfunctional amines containing a small proportion of amines carrying more than two reactive labile hydrogen functions can also be used.

Preferably, tertiary amines forming cationic patterns (a1) are chosen from among compounds corresponding to one of the following formulas:

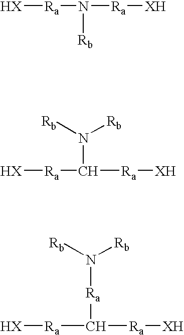

in which

each Rₐ independently represents a linear or ramified alkylene group in C₁₋₆, cycloalkylene in C₃₋₆, or arylene, all of which can be replaced by one or several halogen atoms and can contain one or several heteroatoms chosen from among O, N, P and S,

each R_{b} independently represents an alkyl group in C₁₋₆, cycloalkyl in C₃₋₆, or aryl, all of which can be replaced by one or several halogen atoms and contain one or several heteroatoms chosen from among O, N, P and S, and,

each X independently represents an oxygen or sulphur atom or an NH or NR_{c} group, where R_{c} represents an alkyl group in C₁₋₆.

Preferred tertiary amines for obtaining elastic cationic polyurethanes according to this invention include N-methyldiethanolamine and N-tert-butyldiethanolamine.

Tertiary amines forming cationic patterns (a1) of polyurethanes according to this invention may also be polymers with tertiary amine functions, carrying reactive labile hydrogen functions at their ends. The average molar mass by weight of these polymers with tertiary amine functions is preferably between 400 and 10 000.

Examples of this type of appropriate polymers with amine functions include polyesters derived from polycondensation of N-methyldiethanolamine and adipic acid.

As mentioned above, tertiary amines forming cationic patterns (a1) are partially or totally neutralised by an appropriate neutralisation agent, particularly mineral or organic acids such as hydrochloric acid, hydrobromic acid, carboxylic acids and particularly monocarboxylic acids such as acetic acid, propionic acid, benzoic acid, lactic acid, stearic and oleic acids, and polyacids. The organic acid may possibly carry other functions such as OH (citric acid, salicylic acid).

Neutralisation of tertiary amine functions by an appropriate acid is different from quaternisation of these functions with a quaternisation agent and must not be confused with it.

The second type of patterns forming polyurethanes according to this invention are macromolecular patterns called (a2) patterns, derived from non ionic polymers carrying reactive labile hydrogen functions at their ends and with a vitreous transition temperature (Tg) as measured by differential scanning calorimetry of less than 10° C.

The viscoelastic properties of polyurethanes are particularly attractive when the (a2) patterns are derived from polymers with a vitreous transition temperature of less than 0° C. and even better less than −10° C.

These polymers preferably have an average molar mass by weight between 400 and 10 000, and more particularly between 1000 and 5000.

Non-ionic polymers that can form non-ionic patterns (a2) may be chosen for example from among polyethers, polyesters, polysiloxanes, ethylene and butylene copolymers, polycarbonates and polymers containing fluorine.

In particular, polyethers are preferred and the preferred polyether is poly(tetramethylene oxide).

Diisocyanates forming patterns (b) include aliphatic, alicyclic and aromatic diisocyanates.

The preferred diisocyanates are chosen from among methylenediphenyldiisocyanate, methylenecyclohexanediisocyanate, isophoronediisocyanate, toluenediisocyanate, naphthalenediisocyanate, butanediisocyanate and hexyldiisocyanate. These diisocyanates may obviously be used alone or in the form of a mix of two or several diisocyanates.

As mentioned above, elastic cationic polyurethanes according to this invention may contain a given fraction of patterns (a3) derived from non ionic monomer compounds containing at least two functions with labile hydrogen, in addition to the patterns (a1), (a2) and (b) that are necessarily present in polyurethanes according to this invention.

These patterns (a3) are for example derived from neopentylglycol, hexaethyleneglycol or aminoethanol.

The physical parameter characterising the viscoelastic properties of the above cationic polyurethanes is their coverage in tension. This coverage is determined by tension creep test consisting of quickly stretching a test piece to a pre-determined elongation ratio, and then releasing the stress and measuring the length of the test piece.

The creep test used for characterisation of elastic cationic polyurethanes according to this invention takes place as follows:

The test piece used is a 500±50 mm thick polyurethane film, cut into 80 mm×15 mm strips. This copolymer film is obtained by drying at a temperature of 22±2° C. at a relative humidity of 50±5%, of a solution or dispersion with 3% by weight of the said polyurethane in water and/or ethanol.

Each strip is fixed between two jaws at a distance of 50±1 mm from the other strip, and is stretched at a rate of 20 mm/minute (under the above temperature and relative humidity conditions) to an elongation of 50% ([[Eₘₐₓ]]εₘₐₓ), in other words up to 1.5 times its initial length. The stress is then released by imposing a return rate equal to the stretching rate, namely 20 mm/minute, and the elongation of the test piece (expressed as a percent of the initial length) is then measured immediately after returning to zero load (εᵢ).

The instantaneous coverage (Rᵢ) is calculated using the following formula:
Rᵢ(%)=((εₘₐₓ−εᵢ)/(εₘₐₓ)×100

Elastic cationic polyurethanes according to this invention preferably have an instantaneous coverage (Rᵢ) as measured under the conditions mentioned above of between 5% and 95%, and particularly between 20% and 90% and ideally between 35 and 85%.

The vitreous transition temperature (Tg) of non-ionic polymers forming patterns (a2) and cationic polyurethanes according to this invention is measured by Differential Scanning Calorimetry (DSC) according to ASTM standard D3418-97.

Elastic cationic polyurethanes according to this invention preferably have at least two vitreous transition temperatures, at least one of which is below 10° C., preferably below 0° C. and even better below −10° C., and at least one other is greater than or equal to the ambient temperature (20° C.).

The instantaneous coverage and consequently the viscoelastic properties of polyurethanes according to this invention depend on the fraction of the different monomer patterns (a1), (a2), (a3) and (b).

The fraction of patterns (a1) must be sufficient to give polymers their positive charge responsible for their good affinity for keratinic substrates. Patterns (a2) must represent a sufficient fraction by weight for the polyurethanes to have at least one vitreous transition temperature below 10° C. and that do not form brittle films.

In general, patterns (a1) occupy from 1 to 90%, and preferably from 5 to 60% by weight, patterns (a2) occupy from 10 to 80% and preferably from 40 to 70% by weight, and patterns (a3) occupy from 0 to 50% by weight, and preferably from 0 to 30% by weight of the total polymers.

The quantity of the patterns (b) present is essentially stoichiometric with respect to the sum of the patterns (a1), (a2) and (a3). Obtaining polyurethanes with high molar masses requires a number of isocyanate functions almost identical to the number of functions with labile hydrogen. A person skilled in the subject will know how to choose a molar excess of one particular function type in order to adjust the molar mass to the required value.

As mentioned above, elastic cationic polyurethanes may be incorporated in many cosmetic compositions and they improve their cosmetic properties.

Obviously, the quantity of polyurethane present in the different compositions depends on the type of composition and the required properties and can be varied within a very wide range, usually between 0.5 and 90% by weight, and preferably between 1 and 50% by weight compared with the final cosmetic composition.

When elastic cationic polyurethanes are included in hair lacquer, their concentration is usually between 0.5 and 15% by weight. In nail varnish, they usually represent from 0.5 to 40% of the composition by weight and in make-up foundation compositions, lipsticks and superficial body growths usually contain between 0.5 and 20% by weight of polyurethanes according to this invention.

The use of elastic cationic polyurethanes according to this invention could also be envisaged pure, for example to form a protective film on finger and toe nails.

### EXAMPLE 1

### Synthesis of an Elastic Cationic Polyurethane

The following monomers and solvents are added into a thermostat controlled reaction vessel provided with a mechanical stirring system and a cooler:

1 mole of a mix of diol type monomers, in other words a mix of N-methyldiethanolamine and poly(tetramethylene oxide) with an average molar mass by weight equal to 1400, the molar proportions of each of these two types of monomers being indicated in Table 1 below and,

a quantity of methylethylcetone such that the concentration of diol type monomers is equal to 75% by weight.

The mix is heated to a temperature of 70° C., and then a small molar excess, in other words 1.03 moles of isophoronediisocyanate are added drop by drop while stirring for a duration of about 2 hours. During this addition, the temperature increases until reflux of the solvent.

A sample is taken at regular intervals and an IR absorption spectrum of this sample is plotted to monitor disappearance of the band corresponding to isocyanate functions (2260 cm⁻¹).

When the absorption band of —NCO functions no longer reduces, which usually takes place after about 5 hours, the reactional mix is allowed to cool to ambient temperature, and it is then diluted with acetone such that the concentration of polymer is about 40% by weight.

The next step is to add 20 ml of ethanol to the mix obtained in order to deactivate residual —NCO functions and stirring is continued at ambient temperature until the —NCO functions, in other words the IR absorption band at 2260 cm⁻¹, have completely disappeared.

A sufficient quantity of a hydrochloric acid solution (2 moles/l) is added to neutralise the amine groups to the required rate. The different organic solvents (methylethylcetone, acetone and ethanol) are then eliminated by distillation under a vacuum at a temperature of 40° C.

After eliminating the organic phase, a sufficient quantity of water is added to the aqueous polymer solution to obtain a concentration of polymer in water equal to about 25% by weight.

Three different polyurethanes (PU1, PU2 and PU3) are prepared as described above, with a molar N-methyldiethanolamine/poly(tetramethylene oxide) ratio equal to 2, 3 and 4 respectively.

Table 1 below shows the theoretical molar composition and the physicochemical characteristics of the three polymers obtained.

TABLE 1

| | | | | | Average |
|---|---|---|---|---|---|
| | Pattern | Pattern | Pattern | | molar |
| | (a1)¹⁾ | (a2)²⁾ | (b)³⁾ | Theoretical | mass in |
| | (moles) | (moles) | (moles) | amine index | weight⁴⁾ |
| PU1 | 2 | 1 | 3 | 48 | 36 600 |
| PU2 | 3 | 1 | 4 | 63 | 35 400 |
| PU3 | 4 | 1 | 5 | 75 | 16 800 |

| | | Dry extract | | |
|---|---|---|---|---|
| | | of the | Appearance of | Vitreous |
| | pH of the | aqueous | the aqueous | transition |
| | solution | solution | solution | temperature⁵⁾ |
| PU1 | 3.3 | 23% by weight | clear | −78 and |
| | | | | +30° C. |
| PU2 | 2.1 | 24% by weight | clear | −79 and |
| | | | | +48° C. |
| PU3 | 1.7 | 23% by weight | clear | −80 and |
| | | | | +23° C. |

¹⁾N-methyldiethanolamine
²⁾poly(tetramethylene oxide) with an average mass by weight of 1400, marketed under the name Terathane ® 1400 by the DUPONT Company
³⁾isophoronediisocyanate
⁴⁾measured by chromatography by permeation of gel in THF, detection by refractometry
⁵⁾ASTM D3418-97

### EXAMPLE 2

### Preparation of Capillary Compositions (Lacquers)

Three capillary compositions are prepared, each containing 65 g of dimethylether and 35 g of a water/ethanol mix (1:2) containing 3% by weight of one of the three elastic cationic polyurethanes synthesized in example 1, in an aerosol device.

The compositions obtained can easily be applied to hair.

### EXAMPLE 3

### Evaluation of the Cosmetic Properties of Capillary Compositions

5.4 g, 3 g of each of the hair compositions prepared in example 2 are applied to natural hair, and are allowed to dry for 1 hour.

A panel of 10 persons visually evaluates the cosmetic properties of the hair treated, giving marks varying from 0 to 50 (a mark of 50 is given if the required cosmetic properties are obtained).

The results obtained are summarised in Table 2.

TABLE 2

| | PU1 | PU2 | PU3 |
|---|---|---|---|
| Elasticity | 40 | 40 | 40 |
| Lack of | 50 | 40 | 45 |
| powdering | | | |
| Gloss | 40 | 40 | 40 |
| Softness | 30 | 40 | 40 |
| Lack of | 40 | 45 | 40 |
| stickiness | | | |
| Feel quality | 20 | 30 | 40 |

### EXAMPLE 4

### Measurement of Instantaneous Coverage

Polyurethane films are prepared from dispersions with 3% by weight of each of the polyurethanes in example 1 in a water/ethanol mix (1:2). Table 4 below shows instantaneous coverage values (expressed in %) measured under the following conditions:

film thickness=500±50 mm,

strip dimensions 80 mm×15 mm

drying conditions=22±2° C., relative humidity of 50±5%,

distance between two jaws=50±1 mm,

stretching rate=return rate=20 mm/minute

The instantaneous coverage (Rᵢ) is calculated using the following formula:
Rᵢ(%)=((εₘₐₓ−εᵢ)/εₘₐₓ)×100

TABLE 3

| Polyurethane | Instantaneous coverage |
|---|---|
| PU1 | 40% |
| PU2 | 63% |
| PU3 | 48% |

1. Cosmetic composition containing at least one elastic cationic polyurethane characterised by the fact that they are composed essentially of
(a1) cationic patterns derived from at least one tertiary amine with at least two labile hydrogen reactive functions, the said amine being at least partially neutralised,
(a2) non-ionic patterns derived from non-ionic polymers carrying reactive labile hydrogen functions at their ends with a vitreous transition temperature (Tg) measured by differential scanning calorimetry, less than 10° C., and possibly
(a3) non-ionic patterns derived from non-ionic monomer compounds containing at least two labile hydrogen functions, and
(b) patterns derived from at least one diisocyanate.

2. Cosmetic composition according to claim 1, characterised by the fact that it consists of a hair lacquer and its concentration is between 0.5 and 15% by weight of cationic polyurethane.

3. Cosmetic composition according to claim 1, characterised by the fact that it consists of a nail varnish containing between 0.5 and 40% by weight of cationic polyurethane.

4. Cosmetic composition according to claim 1, characterised by the fact that it consists of a make-up composition for use on the skin, lipsticks and superficial body growth, and that it usually contains between 0.5 and 20% by weight of cationic polyurethane.

5. A hair treatment method comprising applying a hair lacquer to the hair, said hair lacquer comprising elastic cationic polyurethanes according to claim 1.

6. A nail treatment method comprising applying a nail varnish to said nail, said nail varnish comprising elastic cationic polyurethanes according to claim 1.

7. A method of forming a protective film on a finger nail or toe nail, said method comprising applying to said finger nail or toe nail a composition comprising elastic cationic polyurethanes according to claim 1.

8. A cosmetic treatment method comprising applying a cosmetic composition to skin, lips or superficial body growth, said cosmetic composition comprising elastic cationic polyurethanes according to claim 1.
